(19) United States
(12) Patent Application Publication
(10) Pub. No.: US 2024/0389842 A1
(43) Pub. Date: Nov. 28, 2024

(54) ENDOSCOPE SYSTEM & ELEMENTS THEREOF

(71) Applicant: I.Q. ENDOSCOPES LTD, Chepstow, Gwent (GB)

(72) Inventors: Andrew MILLER, Chepstow, Gwent (GB); Patrick WARD-BOOTH, Chepstow, Gwent (GB); James HADLEY, Cambridge, Cambridgeshire (GB)

(21) Appl. No.: 18/691,177

(22) PCT Filed: Sep. 6, 2022
(86) PCT No.: PCT/EP2022/074723

§ 371 Date: Mar. 12, 2024

(30) Foreign Application Priority Data
Sep. 14, 2021 (GB) 2113099.2

(51) Int. Cl. A61B 1/05; A61B 1/00

(52) U.S. Cl. CPC A61B 1/05; A61B 1/00119; A61B 1/00124

(57) ABSTRACT

Various embodiments provide an imaging endoscope system and method for assembling an imaging endoscope system from a kit. The imaging endoscope system comprises a base unit, a hand controller, an umbilical section for releasably connecting the base unit and the hand controller, and an insertion section having a proximal end connected to the hand controller and a distal end for insertion into a subject. The base unit is configured for provision of electrical power and one or more fluids to the insertion section via the umbilical section and the hand controller. The umbilical section comprises an umbilical section connector configured to be releasably engageable with a corresponding base unit connector via a single connection action to make at least one mechanical connection, at least one electrical connection, and at least one fluid connection between the umbilical section connector and the base unit connector during said single connection action.

This application claims priority from GB2113099.2 filed 14 Sep. 2021, the contents and elements of which are herein incorporated by reference for all purposes.

### FIELD OF THE INVENTION

The present invention relates to imaging endoscope systems & elements thereof. It relates particularly to imaging endoscope systems comprising a connection system for connecting an umbilical section of the endoscope to a base unit.

### BACKGROUND

It is known that early detection of disease may be assisted by endoscopic examination of internal structures such as the alimentary canals and airways, e. g., the oesophagus, lungs, colon, uterus, and other organ systems. Endoscopic examination may be carried out using an imaging endoscope. Typically, an imaging endoscope has a flexible tube providing a light guide that guides illumination light from an external light source located proximally of the flexible tube to a distal tip of the flexible tube. The illumination light thereby illuminates the tissue to be examined. The distal tip typically also includes an objective lens to gather light from the tissue being examined. The flexible tube provides an imaging light guide to carry the light away from the distal tip and to a camera at the proximal end of the flexible tube. It is also known for the imaging light guide to be dispensed with and replaced with an imaging camera chip at the distal tip. In this case, the signal from the imaging camera chip is conducted along electrical wires in the flexible tube. Based on either approach, an image is produced for display to the imaging endoscope operator. Typically, it is preferred that the insertion tube is flexible. This allows the insertion tube to be located along tortuous and often complex paths within the body. To allow adequate steering of the flexible tube within the body, it is known to provide the distal tip of the insertion tube with means for deflecting the distal tip, and therefore with means to steer the distal tip. A typical approach to this is to provide control wires, extending along the flexible tube from a control handle at a proximal end of the flexible tube and anchored to the distal tip. The control handle has control knobs to allow movement of the control wires and consequently steering of the distal tip.

As sterility is an important factor for endoscopic procedures, it is known to provide a single reusable base unit for sequential use with several interchangeable identical endoscope components (comprising a hand controller and insertion section as discussed above, for example). These endoscope components may be releasably connectable to the base unit via an umbilical section. One of the primary reasons for adopting this approach is that it is then possible to provide the interchangeable endoscope components in sterile condition at the time of attachment to the base unit. After a single use, the interchangeable endoscope component is intended to be detached from the base unit, disposed of, and the next interchangeable endoscope component, in sterile condition, attached to the base component ready for use. Such an arrangement is disclosed in GB2569013 B—Imaging endoscope system and associated methods, published 9 Sep. 2020, which is herein incorporated by reference.

It is also known, in addition to the imaging function described above, for imaging endoscopes to provide further functionality. For example, it is known for the distal tip to be equipped with a port for dispensing air (or more generally, insufflation gas) to inflate the internal structure being examined, a port for dispensing irrigation liquid such as water or saline. Where such additional functions are to be provided, the base unit may be configured to provide a flow of insufflation gas and/or to provide a flow of irrigation liquid to the distal tip of the endoscope, for by supplying insufflation gas or irrigation liquid along the umbilical section, through the hand controller and along the insertion section to the distal tip. When connecting endoscope components to a base unit which is configured to provide a flow of insufflation gas and/or to provide a flow of irrigation liquid to the distal tip of the endoscope, it is therefore typically necessary to make multiple separate connections (e.g. one for insufflation gas, one for irrigation liquid, and one or more for electrical) between the endoscope component and the base unit. The need to perform multiple connection actions when connecting the endoscope components and the base unit places additional burden on an operator of the endoscope system to ensure that multiple connectors have been correctly connected, and further increases the time required for assembly of the system prior to performing an endoscopy procedure.

The present invention has been devised in light of the above considerations.

### SUMMARY OF THE INVENTION

The present inventors have developed a connection system which allows for facile connection of endoscope components to a base unit, thereby reducing the burden on an operator of the endoscope system to ensure that multiple connectors have been correctly connected, and further reducing the time required for assembly of the system prior to performing an endoscopy procedure.

In a general aspect, the present invention provides a connection system for connecting an umbilical section of an endoscope to a base unit, said connection system comprising an umbilical section connector and a corresponding base unit connector, wherein:
the umbilical section connector is configured to be releasably engageable with the base unit connector via a single connection action to make at least one mechanical connection, at least one electrical connection, and at least one fluid connection between the umbilical section connector and the base unit connector during said single connection action.

Typically, the connection system will be provided as part of an imaging endoscope system. Accordingly, in a first aspect, the present invention provides an imaging endoscope system comprising:
a base unit;
a hand controller;
an umbilical section for releasably connecting the base unit and the hand controller;
an insertion section having a proximal end connected to the hand controller and a distal end for insertion into a subject;
wherein the base unit is configured for provision of electrical power and one or more fluids to the insertion section via the umbilical section and the hand controller, and
wherein the umbilical section comprises an umbilical section connector configured to be releasably engageable with a corresponding base unit connector via a single connection action to make at least one mechanical connection, at least one electrical connection, and at least one fluid connection between the umbilical section connector and the base unit connector during said single connection action.

In a second aspect, the present invention provides a method for assembling an imaging endoscope system from a kit comprising:
a base unit;
a hand controller;
an umbilical section releasably connecting the base unit and the hand controller;
an insertion section having a proximal end connected to the hand controller and a distal end for insertion into a subject;
the base unit being configured for provision of electrical power and one or more fluids to the insertion section via the umbilical section and the hand controller; and
the umbilical section comprising an umbilical section connector configured to be releasably engageable with a corresponding base unit connector;
wherein the method comprises connecting the umbilical section connector and the base unit connector in a single connection action to thereby make at least one mechanical connection, at least one electrical connection, and at least one fluid connection between the umbilical section connector and the base unit connector in said single connection action.

The term ‘single connection action’ here is used to define a single action by an operator of the imaging endoscope system. A single connection action may include manually locating the umbilical section connector and the base unit connector into an engaged position. By providing an arrangement in which at least one mechanical connection, at least one electrical connection, and at least one fluid connection are made between the umbilical section connector and the base unit connector in this single connection action, the time taken for assembly of the system can be greatly reduced in comparison to system where multiple connection actions (for example, connection of multiple connectors) are required to provide both electrical and fluid connections between an endoscope system base unit and other components of the endoscope system. Additionally, the risk of operator error in assembling the system can be reduced, because only a single connection action is required.

To allow for the at least one mechanical connection, at least one electrical connection, and at least one fluid connection to be made between the umbilical section connector and the base unit connector in this single connection action, the connectors may be configured such that at least one mechanical connector portion, at least one electrical connector portion, and at least one fluid connector portion of the umbilical section connector and the base unit connector connect substantially simultaneously, when the umbilical section connector and the base unit connector are engaged with one another. As will be appreciated, the precise configuration of the umbilical section connector and the base unit connector is not particularly limited, as this simultaneous connection may be achieved in a variety of ways. However, one or more preferred or optional features of the connection system and the imaging endoscope system are discussed in greater detail below.

Preferably the umbilical section connector is a unitary composite connector: i.e. it is a single connector providing multiple connection utilities.

Each of the umbilical section connector and the base unit connector may comprise a connector body. The respective shapes of at least a part of the umbilical section connector and at least a part of the base unit connector may be selected to be complementary. This can allow for close-fit engagement of the umbilical section connector and the base unit connector. In some arrangements, the mechanical connection between the umbilical section connector and the base unit connector is provided by a friction-fit connection between a part of the umbilical section connector body and a corresponding part of the base unit connector body.

One of the umbilical section connector and the base unit connector may be a male connector, and the other a female connector. Mechanical connection may be provided between the umbilical section connector and the base unit connector by mating of the male and female connectors.

The umbilical section connector and the base unit connector may be keyed to provide a single connection orientation. In other words, the respective shapes of the umbilical section connector and the base unit connector may be selected to have an order of rotational symmetry of 1, thereby allowing the connectors to engage with one another in only a single connection orientation. This prevents mis-orientated connection of the umbilical section connector and the base unit connector which can reduce the risk of damage to the connectors, and increase speed of connection. The keying of the connectors may be provided in a variety of ways: for example by provision of one or more protrusions of one connector which are configured for engagement with one or more corresponding recesses on the other connector. However, in a preferred arrangement, one of the umbilical section connector and the base unit connector has an engagement part (e.g. a protrusion) having a cross-sectional profile which is a flattened circle, and the other of the umbilical section connector and the base unit connector has a corresponding engagement part (e.g. a recess) also having a flattened circle shape. The term ‘flattened circle’ is used herein to define a generally circular shape, wherein at least one portion of the circle has a radius of curvature larger (i.e. flatter) than another portion of the circular shape. Use of connectors have such a ‘flattened circle’ profile is particularly preferred, because in addition to providing a single rotational connection orientation, a flattened circle shape may be more comfortable for an operator of the endoscope system to hold during connection. Furthermore, connectors having this shape may be relatively easier to clean than e.g. connectors having a number of recesses and/or protrusions to provide a similar keying effect.

At least a part of the umbilical section connector body may be tapered. A tapered portion of the connector body may be more comfortable for an operator of the endoscope system to hold during connection. The diameter of the umbilical section connector may be no greater than 10 cm at its widest point, for example no greater than 9 cm, or no greater than 8 cm. This size ensures that the umbilical section connector can easily be manipulated whilst being held in one hand. In some arrangements the minimum diameter of the umbilical section connector may be e.g. at least 3 cm wide at its widest point, for example at least 5 cm wide. However, it is important to note that the key considerations for the size and shape of the umbilical section connector are that it is configured to be ergonomically comfortable and efficient for a user to hold during use. What that in mind, a wide variety of possible shapes and sizes for the umbilical section connector body are contemplated, and the above dimensions should therefore not be construed as limiting.

The base unit connector may be partially recessed into the base unit to thereby provide an engagement recess configured to receive an engagement portion of the umbilical section connector. The base unit connector body may further comprise a collar portion which extends outwardly from the base unit, and is arranged to surround the umbilical section connector when it is engaged with the base unit connector. This can help to avoid inadvertent disconnection of the connectors.

The system may comprise an interlock member configured to releasably lock the umbilical section connector in an engaged position with respect to the base unit connector. This locking constitutes a mechanical connection between the umbilical section connector and the base unit connector. The interlock member may be biased into a locked position. The precise form and location of the interlock member is not particularly limited, and any suitable interlock member may be used. One example of a suitable interlock member is a latch. A latch is particularly advantageous, as locking of the umbilical section connector and the base unit connector can be achieved in the same single connection action as when the umbilical section connector and the base unit connector are engaged (e.g. during manual location of the umbilical section connector and the base unit connector into an engaged position). The latch may be provided e.g. on a base unit connector body and may be operable to couple to a portion of the umbilical section connector body. In another arrangement, the latch may be provided on a pneumatic connector portion of the base unit, and may be operable to couple to e.g. a recess provided on a pneumatic connector portion of the umbilical section connector. Providing the latch on pneumatic connector portions of the umbilical section connector and base unit connector may be advantageous over other arrangements, as it can help to ensure proper connection of the pneumatic connector portions during engagement of the two connectors. In some arrangements, the latch may be a spring-biased latch, said latch being provided either on the base unit or on the umbilical section connector, and being configured to engage with a component provided on the other of the base unit and the umbilical section connector.

In one preferred arrangement, the interlock member is provided as a spring-biased latch on the base unit, the spring-biased latch being configured to engage with a portion of the umbilical section connector to releasably lock the umbilical section connector in an engaged position with respect to the base unit connector.

The connection system may be configured to produce a sound audible to an operator of the system on coupling of the umbilical section connector and the base unit connector. This audible sound can serve as confirmation to an operator of the endoscope system that the umbilical section connector and the base unit connector are properly engaged. Where the connection system comprises an interlock member, the sound may be produced by operation of the interlock member locking the umbilical section connector in an engaged position with respect to the base unit connector.

The umbilical section connector and base unit connector may be configured to be disengaged by performance of at least one disengagement action that is not an action of manually moving the umbilical section connector away from the base unit connector. This can reduce the risk of accidental disengagement of the connectors.

Where the system comprises an interlock member as described above, the umbilical section connector and base unit connector may be configured to be disengaged by performance of one or more disengagement actions, at least one of said disengagement actions relating to the interlock member. The disengagement action relating to the interlock member may be an action of disengaging the interlock member. The action of disengaging the interlock member may include operating one or more controls provided on the base unit: for example, pressing or more buttons on the base unit. Disengaging the interlock member may be performed mechanically or may be performed in response to an electrical control signal (e.g. from a control module of the base unit).

In some arrangements, at least two disengagement actions may be required. One of these two actions may include manually moving the umbilical section connector away from the base unit connector. In one preferred arrangement, the at least two disengagement actions include a first disengagement action of disengaging the interlock member, and a second disengagement action of moving the umbilical section connector away from the base unit connector.

The first and second disengagement actions may be required to be performed at the same time (i.e. simultaneously) to allow for disengagement of the umbilical section connector and base unit connector. This can further reduce the risk of accidental disengagement of the connectors.

The at least one electrical connection between the umbilical section connector and the base unit connector may be provided by connection of respective electrical contacts provided on the umbilical section connector and the base unit connector. The precise nature of the electrical contacts is not particularly limited: any suitable electrical contacts may be used. However, it is typically necessary to make multiple electrical connections between the base unit and the umbilical section of the endoscope, e.g. to provide various different electrical functions such a provision of power or transmission of electrical signals between the base unit and other endoscope components. Accordingly, the umbilical section connector may comprise a first array of electrical contacts configured for electrical engagement with a corresponding second array of electrical contacts provided on the base unit connector.

In some embodiments, the respective electrical contacts provided on the umbilical section connector and the base unit connector may be provided by USB-type connections: e.g. male and female USB-type connections. For example, the umbilical section connector may comprise a female USB connector, and the base unit may comprise a male USB connector, or vice versa. The USB connection may be a USB-A, USB-B or USB-C type connection. Advantageously, use of USB-type connectors can reduce manufacturing complexity of the system, as such connectors are readily available as ‘off-the-shelf’ component parts.

In some embodiments, one of the first and second arrays of electrical contacts may comprise spring-loaded pins (sometimes referred to as ‘pogo pins’). The other of the first and second arrays of electrical contacts may comprise terminal pads e.g. on a printed circuit board (PCB). In some arrangements, each of the first and second array of electrical contacts may comprise both spring-loaded pins and terminal pads. However, in preferred arrangements, the second array of electrical contacts (on the base unit connector) comprises spring-loaded pins, and the first array of electrical contacts comprises (on the umbilical connector) comprises terminal pads of a PCB.

Pogo pins can offer improved durability over other electrical contacts and can also provide electrical connection with relatively high level of resilience to mechanical shock and vibration. Furthermore, the use of pogo pins allows for an electrical connection to be provided between the umbilical section connector and the base unit connector, whilst still allowing for some tolerance in the exact relative locations of the two connectors. This is particularly advantageous in the context of the present invention, where multiple different types of connection are required to be made in a single connection action, as it can reduce the risk of forming an incomplete electrical connection on engagement of the umbilical section connector and the base unit connector.

The first and second arrays of electrical contact may be arranged in any suitable manner, for example, in a grid array, or in a line. In one preferred arrangement, the first and second arrays of electrical contacts are arranged in a substantially semi-circular array. As there is often a minimum required distance between adjacent electrical contacts required to meet operational standards, providing the electrical contacts in a substantially semi-circular array allows for these minimum required distances to be met whilst reducing or minimising overall surface area taken up by said electrical contacts.

The electrical contacts of the first and second arrays of electrical contacts may be substantially symmetrically distributed on an engagement face of their respective connector. For example, an even number of electrical contacts may be provided on a left side of said engagement face as compared with a right side of the engagement face. Providing a symmetrical distribution of electrical contacts allows for an even distribution of pressure on the contacts when the umbilical section connector and the base unit connector are arranged in an engaged position. This can minimise wear on the electrical contacts over time, thereby prolonging the lifetime of the connectors.

The at least one fluid connection may be provided by respective umbilical connector and base unit connector pneumatic connector portions configured to allow supply of liquids and/or gases from the base unit to the umbilical section when the umbilical section connector and the base unit connector are in an engaged position. Any suitable pneumatic connectors may be used. One of the pneumatic connector portions may be a male connector, and the other a corresponding female connector. Examples of suitable pneumatic connectors are Nordson Medical 20AC-PB3-01 (male) and 20CBV-SB3-02 (female) connectors. The pneumatic connector portions may be mainly formed from a polymeric material. Alternatively, they may be mainly formed from metal. Polymeric components are generally cheaper to produce but may have a shorter lifespan than metallic components. In preferred arrangements, the pneumatic connector portion provided in the umbilical connector is constructed mainly of plastic, and the pneumatic connector portion provided in the base unit is constructed mainly of metal. This arrangement is preferred for systems comprising a single reusable base unit for sequential use with several interchangeable identical endoscope components releasably connectable to the base unit via an umbilical section, because the umbilical section of the single-use disposable endoscope can be provided at low cost, whilst the base unit pneumatic connector has a longer lifespan.

The system may be configured to provide a flow of insufflation gas to a region of tissue of interest during an endoscopy procedure. Accordingly, the base unit may comprise a source of insufflation gas. For example, the base unit may comprise an internal compressed gas cylinder or other gas source. Alternatively or additionally, a separate source of insufflation gas may be provided, external to the base unit. The base unit and/or the umbilical section may be fluidically connected to such an external source of insufflation gas. For example, the base unit may be configured for connection to an external compressed gas cylinder, to a hospital gas supply, or to another suitable external gas source.

The insufflation gas is preferably carbon dioxide, CO₂. CO₂ is particularly preferred for use as an insufflation gas as it is non-flammable, colourless and has a higher blood solubility than air.

Control of gas delivery may be provided by provision of a mechanical ‘trumpet’ valve in the hand controller of the endoscope system. In such an arrangement, the insufflation gas may be supplied at a suitable fixed pressure, and the timing of delivery and the rate of delivery to the distal end of the endoscope may then be controlled by the user operating the mechanical valve at the hand controller in an analogue manner (allowing different flow rates of insufflation gas based on how far they the valve is pressed by the user). There may optionally be provided one or more insufflation gas check valves and/or pressure control valves to control the supply of insufflation gas to be at a suitable fixed pressure. Said valves may be provided in the base unit, or at a gas source outlet.

Alternatively, control of gas delivery may be provided by an insufflation gas valve provided in the base unit, control of the insufflation gas valve controlling the flow of insufflation gas. In such arrangements, the hand controller may have an insufflation gas electronic control switch, operation of the insufflation gas electronic control switch at the hand controller thereby controlling the insufflation gas valve.

The system may be configured to provide a flow of irrigation liquid to a region of tissue of interest during an endoscopy procedure. Accordingly, the system may comprise a source of irrigation liquid. For example, the base unit may comprise an internal irrigation liquid reservoir. Alternatively or additionally, a separate source of irrigation liquid may be provided, external to the base unit. The base unit and/or the umbilical section may be fluidically connected to such an external source of irrigation liquid. For example, the base unit and/or umbilical section may be configured for connection to an external irrigation liquid reservoir, or to another suitable external liquid source.

The base unit may comprise means for pressuring the source of irrigation liquid, such that the irrigation liquid can be supplied at a suitable fixed pressure to the umbilical section. In some arrangements, means for pressuring the source of irrigation liquid may be the same as the source of insufflation gas. For example, where the source of insufflation gas comprises a compressed gas cylinder, the same compressed gas cylinder may be used to pressurise the source of irrigation liquid to assist in delivery of the irrigation liquid.

Where the base unit is configured for connection to an external source of irrigation liquid, the irrigation liquid may be supplied to the umbilical section via the base unit. Alternatively, the irrigation liquid may be supplied directly from the external source of irrigation liquid to the umbilical section.

Control of irrigation liquid delivery may be provided by provision of a mechanical ‘trumpet’ valve in the hand controller of the endoscope system. In such an arrangement, the irrigation liquid may be supplied at a suitable fixed pressure, and the timing of delivery and the rate of delivery to the distal end of the endoscope may then be controlled by the user operating the mechanical valve at the hand controller in an analogue manner (allowing different flow rates of irrigation liquid based on how far the valve is pressed by the user).

Alternatively, control of irrigation liquid may be provided by an irrigation liquid valve provided in the base unit, control of the irrigation liquid valve controlling the flow of irrigation liquid. In such arrangements, the hand controller may have an irrigation liquid electronic control switch, operation of the irrigation liquid electronic control switch at the hand controller thereby controlling the irrigation liquid valve.

In one particularly preferred arrangement, the endoscope system comprises a pressurized gas source (e.g. a compressed gas cylinder) and an irrigation liquid reservoir (e.g. a water bottle), and the umbilical section connector and the base unit connector are configured such that when they are in an engaged position, at least two fluid flow paths are provided:
(i) a first fluid flow path defined between the pressurized gas source and the distal end of the insertion section, for delivery of insufflation gas from the pressurized gas source to a region of tissue of interest during an endoscopy procedure. This first fluid flow path may extend from the base unit, along the umbilical section, through the hand controller and along the insertion section; and
(ii) a second fluid flow path defined between the pressurized gas source and the irrigation liquid reservoir, such that the irrigation liquid reservoir is pressurised by the pressurized gas source.

The present inventors consider that this arrangement is particularly advantageous for reasons discussed below, and may furthermore be applicable even to systems where the umbilical section connector and the base unit connector require more than a single connection action for engagement (i.e. this arrangement may have utility in systems where multiple connection actions are necessary to make at least one mechanical connection, at least one electrical connection, and at least one fluid connection between the umbilical section connector and the base unit connector). Accordingly, this arrangement can be considered to be a further aspect of the present invention.

In this arrangement, a single pressurized gas source can be used both for supply of insufflation gas to a distal end of the insertion section, and also for pressurizing of an irrigation liquid reservoir, such that irrigation liquid can be supplied from the irrigation liquid reservoir at a suitable fixed pressure. Accordingly, a third fluid flow path may also be provided, defined between the irrigation liquid reservoir and the distal end of the insertion section, for delivery of irrigation liquid from the irrigation liquid reservoir to a region of tissue of interest during an endoscopy procedure. This third fluid flow path may extend from the irrigation liquid reservoir, via the umbilical connector, along the umbilical section, through the hand controller and along the insertion section. Alternatively, this third fluid flow path may extend directly from the irrigation liquid reservoir to the hand controller, and along the insertion section. Such arrangements allow the endoscope system to supply both insufflation gas and irrigation liquid to a distal end of the insertion section in a relatively simple and low-cost system, because it is not necessary to provide a separately pressurized irrigation liquid supply.

At least a part of the first and second fluid flow paths may be the same. For example, a single flow path may be provided between the pressurized gas source and the umbilical section connector, and the umbilical section connector may comprise a flow path splitter (such as a ‘T-piece’ component) which splits the single flow path into the first and second fluid flow paths defined above.

The system may be configured to provide suction to a region of tissue of interest during an endoscopy procedure, via the insertion section. The system may therefore further comprise a vacuum source. A suitable vacuum source may be a vacuum bottle. A vacuum tube or conduit may therefore be provided, extending from a distal end of the insertion section, through the hand controller, to the umbilical section connector, wherein the umbilical section connector is configured to be releasably connectable to the vacuum source.

Control of the applied suction may be provided by provision of a mechanical ‘trumpet’ valve in the hand controller of the endoscope system. In such an arrangement, a vacuum may be applied by the vacuum source at a suitable fixed pressure. The strength of applied suction may then be controlled by the user operating the mechanical valve at the hand controller in an analogue manner based on how far the valve is pressed by the user.

Further optional features of the imaging endoscope system (not specifically related to the connection system for connecting an umbilical section of an endoscope to a base unit) will now be discussed.

The umbilical section may be non-releasably connected to the hand controller. The umbilical section may accordingly be integral with the hand controller. This allows for additional simplification of the assembly of the imaging endoscope system.

The distal end of the insertion section may comprise a distal tip assembly located at the distal end of the insertion section and a steering section located adjacent and proximal to the distal tip.

The distal tip assembly may include a light source for illumination of a region of tissue of interest. Preferably, the light source includes at least one light emitting diode (LED). There may be provided at least one electrical conductor along the insertion section for providing electrical power to the light source.

The distal tip assembly may further include an imaging chip for imaging the region of tissue of interest. There may be provided at least one electrical conductor along the insertion section for providing electrical power to the imaging chip. There may be provided at least one electrical conductor for conducting electrical signals from the imaging chip to the proximal end of the insertion section. In this manner, it is possible to implement an imaging endoscope system in which there is no need for the insertion section to transmit light, whether as illumination or optical images.

The distal tip may include a distal tip housing comprising a light-transmissive portion. The distal tip housing may be integrally formed from a polymeric material.

The distal tip housing may have a collar portion extending proximally of a distal end face. The collar portion may be adapted to fit over a distal end of the steering section. This is considered to be a convenient implementation.

The distal tip housing may have a cleaning nozzle arranged at the distal end face, to direct irrigation liquid to clean a lens of the imaging chip.

The steering section may be bendable for steering by operation of the hand controller. The hand controller may include at least one steering control for controlling bending of the steering section.

The insertion section may comprise at least one steering wire, fixed at or near the distal end of the insertion section, and extending along the length of the insertion section for connection to the hand controller, whereby the steering section of the insertion section is bendable for steering by application of tension to the at least one steering wire by operation of the hand controller. There may be provided such four steering wires, the four steering wires being substantially equiangularly spaced around the insertion section when viewed in cross section.

The steering wires may have sheaths each defining an axis of constrained movement for each respective steering wire. In other words, preferably the steering wires are provided as Bowden cables. Preferably, the steering wires extend through the steering section to the distal tip and the sheaths of the steering wires do not extend through the steering section. In this way, application of tension to the one or more steering wires can cause bending primarily in the steering section of the insertion section, without substantially influencing the curvature of the remaining portion of the insertion section intermediate the hand controller and the steering section.

The steering section may comprise a series of links pivotably connected to each other, or connected to one another via living hinges, such that the links are arranged to cooperate to permit controlled bending of the steering section. The connections between each link may provide a limit to the range of relative movement between adjacent links. The links may have apertures dimensioned to receive and conduct the steering wires.

Other features of the imaging endoscope, or imaging endoscope system may be implemented as described in GB2569013 B—Imaging endoscope system and associated methods, published 9 Sep. 2020, which is herein incorporated by reference.

The imaging endoscope may be used in gastroscopy, colonoscopy, or any other suitable imaging application. In preferred arrangements, the imaging endoscope is a gastroscope or a colonoscope.

### SUMMARY OF THE FIGURES

Embodiments and experiments illustrating the principles of the invention will now be discussed with reference to the accompanying figures in which:

FIG. 1 shows a schematic view of an imaging endoscope system according to an embodiment of the present invention.

FIG. 2 is a perspective line drawing of an umbilical section connector forming a part of an imaging endoscope system according to an embodiment of the present invention.

FIG. 3 is a schematic partial cross-sectional illustration of the umbilical section connector of FIG. 2.

FIG. 4 is a perspective line drawing of a portion of a base unit forming a part of an imaging endoscope system according to an embodiment of the present invention, showing in particular the base unit connector.

FIG. 5 is a cross-section showing the interaction between the umbilical section connector and the base unit connector of a connection system according to the present invention.

### DETAILED DESCRIPTION OF THE INVENTION

As set out above, the present inventors have developed a connection system which allows for facile connection of endoscope components to a base unit, thereby reducing the burden on an operator of the endoscope system to ensure that multiple connectors have been correctly connected, and further reducing the time required for assembly of the system prior to performing an endoscopy procedure.

Aspects and embodiments of the present invention will now be discussed with reference to the accompanying figures. Further aspects and embodiments will be apparent to those skilled in the art. All documents mentioned in this text are incorporated herein by reference.

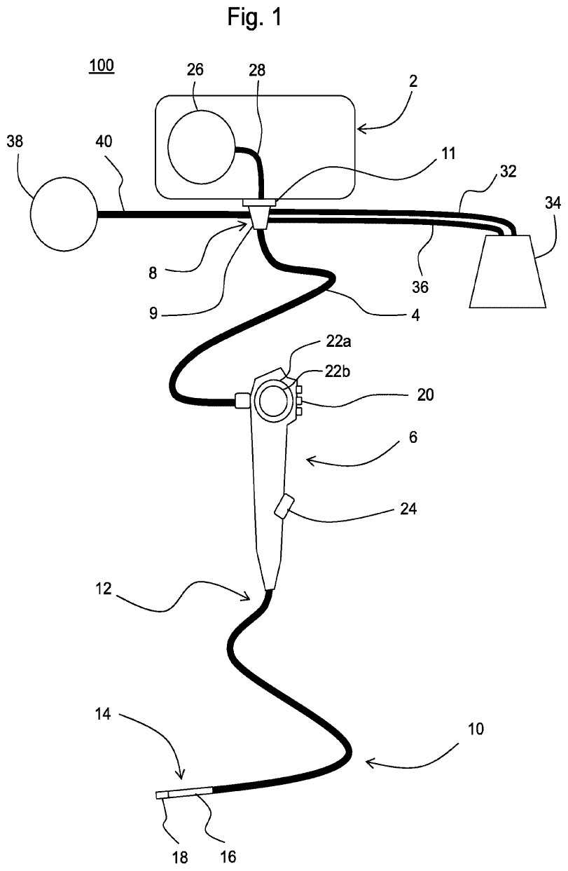

FIG. 1 shows a schematic view of an imaging endoscope system 100 according to an embodiment of the present invention. The system includes a base unit 2 that is intended to be re-usable. Umbilical section 4 releasably connects the base unit 2 and hand controller 6 via a connection system 8 comprising an umbilical section connector 9 and a corresponding base unit connector 11, which is discussed in greater detail below. Insertion section 10 has a proximal end 12 connected to the hand controller 6 and a distal end 14 for insertion into a subject. The distal end 14 has a steering section 16 and a distal tip assembly 18, which will be described in greater detail later.

The hand controller comprises buttons 20 operable to control mechanical valves for control of delivery of insufflation gas, irrigation liquid and suction via the umbilical section 4 and hand controller 6 to the insertion section 10 in a manner discussed in greater detail below.

The hand controller 6 further includes a steering control mechanism including first and second steering wire controls 22a, 22b, for controlling bending of the steering section 16. The first and second steering wire controls 22a, 22b are, in this arrangement, provided as first and second rotatable control wheels. The first and second steering wire controls 22a, 22b are operable by a user to apply tension to four steering wires (not shown) in order to control the bending of the steering section 16 in a known manner: rotation of the first rotatable control wheel winds a first pair of steering wires on or off a rotational drive wheel to thereby bend the steering section in a first plane, and rotation of the second rotatable control wheel winds a second pair of steering wires on or off a rotational drive wheel to thereby bend the steering section in a second plane, perpendicular to the first plane.

The steering wires (not shown) are provided as part of a Bowden cable, i.e. each wire is surrounded by a sheath defining an axis of constrained movement for each respective steering wire. The steering wires extend along the length of the insertion section 10, through the steering section 16 to the distal tip assembly 18. The sheaths of the steering wires do not extend through the steering section 16. In this way, application of tension to the steering wires can cause bending primarily in the steering section of the insertion section, without substantially influencing the curvature of the remaining portion of the insertion section intermediate the hand controller and the steering section.

The hand controller further comprises a tool insertion port 24 adapted to receive one or more surgical instruments, such as biopsy forceps.

The distal end 14 of the insertion section includes the steering section 16 and the distal tip assembly 18. An outer casing component is provided to encase the distal end of the insertion section. This outer casing component may be e.g. a polymeric sheath. The distal tip assembly 18 includes a light source (not shown) provided within a lighting and imaging housing portion on the distal end face of the distal tip assembly, for illumination of a region of tissue of interest. Typically this light source is an LED. At least one electrical conductor (not shown) is provided along the insertion section 10 for providing electrical power to the light source. The distal tip assembly 18 further includes an imaging chip (not shown) provided within a lighting and imaging housing portion for imaging the region of tissue of interest. The imaging chip is provided in the form of a camera, with an objective lens located to collect and direct light onto the imaging chip. An electrical conductor (not shown) is provided for conducting electrical signals from the imaging chip to the proximal end 12 of the insertion section 10. The distal tip assembly 18 further comprises a cleaning nozzle (not shown) arranged at the distal end face, to direct irrigation liquid to clean the lighting and imaging housing portion during use.

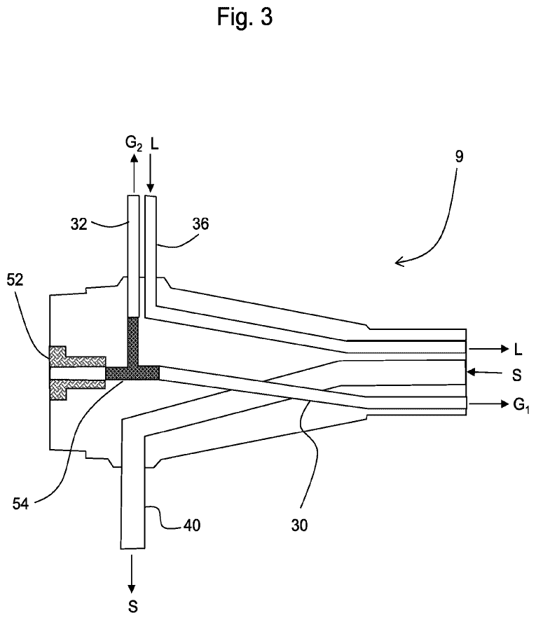

The system is configured to provide a flow of insufflation gas to a region of tissue of interest during an endoscopy procedure. Accordingly, the base unit comprises a source of insufflation gas 26—here, conveniently provided as an internal compressed gas cylinder located inside the base unit 2. The source of insufflation gas is connected to the base section connector 11 via a first gas line 28. When the umbilical section connector and the base unit connector are in an engaged position, this first gas line connects to further gas lines 30, 32 within the umbilical section connector, as shown in FIG. 3. Operation of the system to provide a flow of insufflation gas to a region of tissue of interest during an endoscopy procedure will be discussed in further detail below.

The system is configured to provide a flow of irrigation liquid to a region of tissue of interest during an endoscopy procedure. Accordingly, the system comprises an irrigation liquid reservoir 34, here provided as an external irrigation liquid reservoir. The irrigation liquid reservoir is attached to other components of the system via two fluid flow lines. One of the fluid flow lines is a further gas line 32 as discussed briefly above, which is fluidly connected to the source of insufflation gas 26 via the connection system 8, and the first gas line 28. As the source of insufflation is a compressed gas cylinder, this thereby pressurizes the irrigation liquid reservoir 34. This allows for the irrigation liquid to be supplied at a suitable fixed pressure to the umbilical section 4 via the second fluid flow line which is an irrigation liquid line 36, extending from the irrigation reservoir to the umbilical section connector 11. Operation of the system to provide a flow of irrigation liquid to a region of tissue of interest during an endoscopy procedure will be discussed in further detail below.

The system is also configured to provide suction to a region of tissue of interest during an endoscopy procedure, via the insertion section. The system comprises a vacuum source 38 which is typically provided as a vacuum bottle as already known for medical applications. Vacuum line 40 extends from vacuum source 38 to the umbilical section connector 9 part of the connection system 8. Control of suction created by the vacuum source will be discussed in greater detail below.

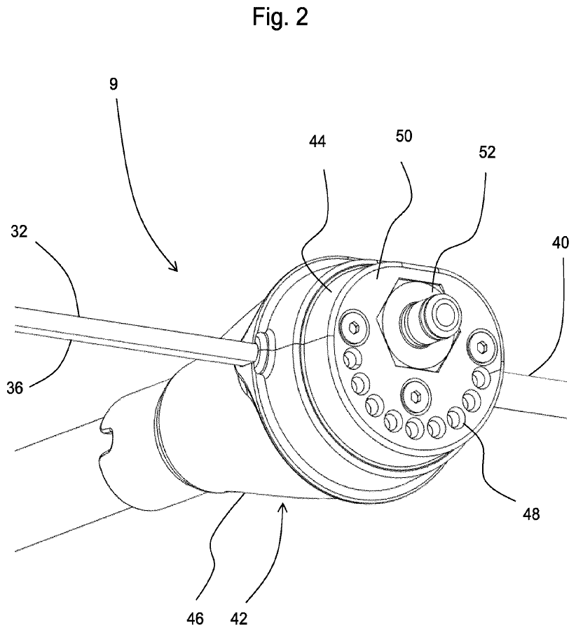

The connection system 8 will now be described in greater detail with reference to FIGS. 2-5. As mentioned above, the connection system is configured to allow releasable connection of the umbilical section 4 to the base unit 2. The connection system comprises an umbilical section connector 9 and a corresponding base unit connector 11.

FIG. 2 is a perspective line drawing of the umbilical section connector 9. The umbilical section connector 9 is a unitary composite connector configured to be releasably engageable with the base unit connector 11 via a single connection action to make at least one mechanical connection, at least one electrical connection, and at least one fluid connection between the umbilical section connector and the base unit connector during said single connection action. In the arrangement shown, the single connection action is an action of manually locating the umbilical section connector 9 and the base unit connector 11 into an engaged position.

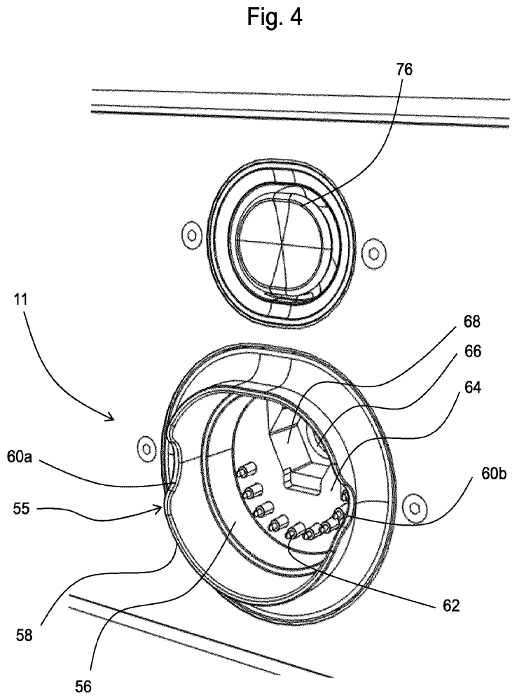

The umbilical section connector 9 is a male connector having a connector body 42, the shape of at least a part of which (an ‘engagement portion’ 44 of the connector) is selected to be complementary to the shape of at least a part of the base unit connector (shown in FIG. 4, which is a perspective line drawing of a portion of the base unit including the base unit connector) to allow for close-fit engagement of the umbilical section connector 9 and the base unit connector 11. The engagement portion 44 of the umbilical section connector has a cross-sectional profile which is a flattened circle. This allows for a keying interaction between the umbilical section connector and the base unit connector, which comprises a corresponding recess also having a flattened circle shape. The shape of the umbilical section connector 9 means that it has a rotational symmetry of order 1, such that there is only a single connection orientation in which the umbilical section connector and the base unit connector will connect. This prevents mis-orientated connection of the umbilical section connector and the base unit connector which can reduce the risk of damage to the connectors, and increase speed of connection.

At least a part of the umbilical section connector body 42 is a tapered portion 46. A tapered portion of the connector body may be more comfortable for an operator of the endoscope system to hold during connection. The diameter of the umbilical section connector is about 8 cm wide at its widest point, which ensures that the umbilical section connector can easily be manipulated by an operator of the endoscope system whilst being held in one hand.

The umbilical section connector comprises a PCB (not shown) having a semi-circular array of terminals 48. The terminals are disposed within recesses provided on an engagement face 50 of the umbilical section connector. These terminals are configured for engagement with corresponding electrical contacts provided on the base unit, as discussed below. The PCB terminals are substantially symmetrically distributed about a mid-line of the engagement face 50 of the umbilical section connector.

The umbilical section connector comprises a pneumatic connector portion 52, which is here conveniently provided as a Nordson Medical 20AC-PB3-01 (male) pneumatic connector, and which is configured for engagement with a corresponding female pneumatic connector provided by the base unit connector. The pneumatic connector is formed from plastic. This allows the umbilical section connector to be produced with a relatively low overall component cost.

The vacuum line 40, gas line 32 and irrigation line 36 each pass through ports formed in the umbilical section connector body 42. The configuration of these lines within the connector is shown in FIG. 3, which is a schematic partial cross-sectional illustration of the umbilical section connector (only part of the pneumatic connector portion 52 is visible in this figure).

Vacuum line 40 passes from the vacuum source 38 (not shown in FIG. 3), through a side port of the umbilical section connector, and onwards to distal end of the insertion section via suitable conduits provided in the umbilical section, hand controller and the insertion section (not shown here). During use of the system, suction is applied by the vacuum source at a suitable fixed pressure, and the strength of applied suction along suction flow path S is provided by operation of one of buttons 20 which controls a mechanical ‘trumpet’ valve in the hand controller of the endoscope system in an analogue manner based on how far the button is pressed by the user.

The pneumatic connector portion (partly shown) is connected to a flow path splitter 54 which is a ‘t-piece’ component arranged to divide the single flow path extending from the pneumatic connector portion 52 into first and second fluid flow paths. The first and second fluid flow paths extend along the further gas lines 30, 32 discussed previously. In this way, when the umbilical section connector and the base unit connector are engaged position, the following gas flow paths are provided:
(i) a first gas flow path G₁ (the direction of which is indicated by arrows in FIG. 3) defined between the source of insufflation gas 26 (which is a compressed gas cylinder) and the distal end of the insertion section, for delivery of insufflation gas to a region of tissue of interest during an endoscopy procedure, said first gas flow path extending through first gas line 28, via pneumatic connector portions of the umbilical section connector and base unit connector, via flow path splitter 54, and along further gas line 30, onwards to distal end of the insertion section via suitable conduits provided in the umbilical section, hand controller and the insertion section; and
(ii) a second gas flow path G₂ (the direction of which is indicated by arrows in FIG. 3) defined between the source of insufflation gas 26 (which is a compressed gas cylinder) and the irrigation liquid reservoir 34 such that the irrigation liquid reservoir is pressurised by the pressurized gas source, said second gas flow path extending through first gas line 28, via pneumatic connector portions of the umbilical section connector and base unit connector, via flow path splitter 54, and along further gas line 32, onwards to the irrigation liquid reservoir 34.

During use of the system, control of insufflation gas delivery along gas flow path G₁ is provided by operation of one of buttons 20 which controls a mechanical ‘trumpet’ valve in the hand controller of the endoscope system in an analogue manner, thereby allowing different flow rates of insufflation gas based on how far the button is pressed by the user.

Irrigation line 36 passes from the irrigation reservoir (not shown in FIG. 3), through a side port of the umbilical section connector, and onwards to distal end of the insertion section via suitable conduits provided in the umbilical section, hand controller and the insertion section (not shown here). In this arrangement, irrigation liquid is supplied along liquid flow path L (the direction of which is indicated by arrows in FIG. 3) at a suitable fixed pressure as a result of pressurization of the irrigation liquid reservoir by the compress gas cylinder via gas line 32. During use of the system, control of irrigation liquid delivery is provided by operation of one of buttons 20 which controls a mechanical ‘trumpet’ valve in the hand controller of the endoscope system in an analogue manner, thereby allowing different flow rates of irrigation liquid based on how far the button is pressed by the user.

FIG. 4 is a perspective line drawing of a portion of the base unit including the base unit connector 11. The base unit connector comprises a base unit connector body 55. The base unit connector body is partially recessed into the base unit to thereby provide an engagement recess 56 configured to receive the engagement portion 44 of the umbilical section connector. As described above, the engagement recess 56 has a flattened circle shape, thereby allowing for keyed engagement of the base unit connector and the umbilical section connector. The base unit connector body 55 further comprises a collar portion 58 which extends outwardly from the base unit, and is arranged to surround the umbilical section connector when it is engaged with the base unit connector. This can help to avoid inadvertent disconnection of the connectors. The collar portion 58 comprises two cut-out potions 60a, 60b, which are arranged to accommodate vacuum line 40, gas line 32 and irrigation line 36 as they extend from the umbilical section connector body.

The base unit connector 11 comprises a semi-circular array of pogo pin electrical contacts 62. The pogo pins 62 are disposed on an engagement face 64 of the base unit connector. These pogo pins 62 are configured for engagement with corresponding electrical contacts provided on the umbilical section connector, as discussed above. The pogo pins are substantially symmetrically distributed about a mid-line of the engagement face 64 of the base unit connector. The use of pogo pins as the base unit connector electrical contacts can also provide electrical connection with relatively high level of resilience to mechanical shock and vibration. Furthermore, the use of pogo pins allows for an electrical connection to be provided between the umbilical section connector and the base unit connector whilst still allowing for some tolerance in the exact relative locations of the two connectors. This is particularly advantageous in the context of the present invention, where multiple different types of connection are required to be made in a single connection action, as it can reduce the risk of forming an incomplete electrical connection on engagement of the umbilical section connector and the base unit connector.

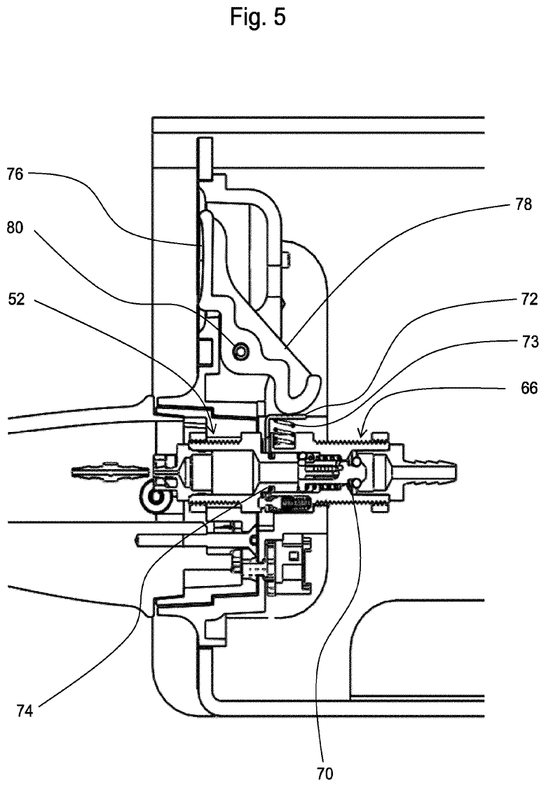

The base unit connector comprises a pneumatic connector portion 66, which is here situated in a recess 68. The base unit connector portion is conveniently provided as a Nordson Medical 20CBV-SB3-02 (female) pneumatic connector, and which is configured for engagement with a corresponding male pneumatic connector portion 52 of the umbilical section connector, as shown in greater detail in FIG. 5. The pneumatic connector is constructed mainly from metal, thereby giving good durability for multiple sequential connections of the base unit connector with an umbilical section connector.

FIG. 5 is a cross-section through a part of the base unit, showing the interaction between the umbilical section pneumatic connector portion 52, and the base unit pneumatic connector portion 66. When the umbilical section pneumatic connector portion 52 is inserted into the base unit pneumatic connector portion 66, it opens spring-biased valve 70, thereby allow for fluid communication between the pneumatic connector portions, and thereby also allowing for fluid connection between first gas line 38 (not shown) and further gas lines 30, 32 (also not shown).

A spring-biased latch 72 is also provided, which acts as an interlock member operable to lock the umbilical section pneumatic connector portion 52, and the base unit pneumatic connector portion 66 in an engaged position. The latch is provided on the base unit pneumatic connector portion 66, and is operable to couple to a recess 74 provided on a pneumatic connector portion of the umbilical section connector: specifically, a portion of the pneumatic connector portion 52 passes through a hole in the latch, and the upwards biasing force of the spring 73 on the latch results in engagement of a bottom edge of said hole with the recess 74. The biasing force provided by spring 73 ensures that the latch 72 is biased into a locked position, thereby helping to prevent accidental disengagement of the connectors.

An audible click may be heard on coupling of the umbilical section pneumatic connector portion 52 and the base unit pneumatic connector portion 66, resulting from engagement of the spring-biased latch. This audible click can serve as confirmation to an operator of the endoscope system that the umbilical section connector and the base unit connector are properly engaged.

The spring-biased latch is arranged to be unengaged by user operation of a control provided on the base unit: here, by pressing button 76 provided on the base unit (also shown in FIG. 4). In the arrangement shown, pressing button 76 causes arm 78 to pivot about pivot point 80, depressing and thereby mechanically disengaging the spring-biased latch 72 from recess 74.

In this way, it is required to perform an action other than manually moving the umbilical section connector away from the base unit connector in order to disengage the connectors. Specifically, disengagement of the connectors requires at least a first disengagement action of pressing button 76 to mechanically disengage the spring-biased latch 72. In some cases, the spring force provided by the spring-biased latch will be sufficient to ‘eject’ the umbilical section connector upon performance of this first disengagement action, resulting in movement of the umbilical section connector away from the base unit connector and disengagement of the connectors. However, in some other arrangements, a second disengagement action of moving the umbilical section connector away from the base unit connector will be required. The first and second disengagement actions may be performed at the same time: i.e., the button 76 can be held whilst moving the umbilical section connector away from the base unit connector. This can significantly reduce the risk of accidental disengagement of the connectors during use of the endoscope system.

***

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example +/−10%.

1. An imaging endoscope system comprising:
a base unit;
a hand controller;
an umbilical section for releasably connecting the base unit and the hand controller;
an insertion section having a proximal end connected to the hand controller and a distal end for insertion into a subject;
wherein the base unit is configured for provision of electrical power and one or more fluids to the insertion section via the umbilical section and the hand controller, and
wherein the umbilical section comprises an umbilical section connector configured to be releasably engageable with a corresponding base unit connector via a single connection action to make at least one mechanical connection, at least one electrical connection, and at least one fluid connection between the umbilical section connector and the base unit connector during said single connection action.

2. The imaging endoscope system according to claim 1 wherein the umbilical section connector and the base unit connector are configured such that at least one mechanical connector portion, at least one electrical connector portion, and at least one fluid connector portion of the umbilical section connector and the base unit connector connect substantially simultaneously, when the umbilical section connector and the base unit connector are engaged with one another.

3. The imaging endoscope system according to claim 1 wherein one of the umbilical section connector and the base unit connector is a male connector, the other is a female connector, and wherein mechanical connection is provided between the umbilical section connector and the base unit connector by mating of the male and female connectors.

4. The imaging endoscope system according to claim 1 wherein the umbilical section connector and the base unit connector are keyed to provide a single connection orientation.

5. The imaging endoscope system according to claim 4 wherein one of the umbilical section connector and the base unit connector has a part having a cross-sectional profile which is a flattened circle, and the other of the umbilical section connector and the base unit connector has a corresponding recess also having a flattened circle shape.

6. The imaging endoscope system according to claim 1 wherein the system comprises an interlock member configured to releasably lock the umbilical section connector in an engaged position with respect to the base unit connector.

7. The imaging endoscope system according to claim 6 wherein the interlock member comprises a spring-biased latch.

8. The imaging endoscope system according to claim 1 wherein the umbilical section connector and base unit connector are configured to be disengaged by performance of at least one disengagement action that is not an action of manually moving the umbilical section connector away from the base unit connector.

9. The imaging endoscope system according to claim 1 wherein the umbilical section connector comprises a first array of electrical contacts configured for electrical engagement with a corresponding second array of electrical contacts provided on the base unit connector.

10. The imaging endoscope system according to claim 9 wherein one of the first and second arrays of electrical contacts comprises spring-loaded pins, and the other of the first and second arrays of electrical contacts comprises terminal pads on a printed circuit board (PCB).

11. (canceled)

12. The imaging endoscope system according to claim 9 wherein the electrical contacts of the first and second arrays of electrical contacts are substantially symmetrically distributed on an engagement face of their respective connector.

13. The imaging endoscope system according to claim 1 wherein the base unit comprises a pressurized gas source.

14. The imaging endoscope system according to claim 1 wherein the system comprises an irrigation liquid reservoir.

15. The imaging endoscope system according claim 14 wherein the base unit comprises means for pressuring the irrigation liquid reservoir.

16. The imaging endoscope system according to claim 1 wherein the endoscope system comprises a pressurized gas source and an irrigation liquid reservoir, and the umbilical section connector and the base unit connector are configured such that when they are in an engaged position, at least two fluid flow paths are provided:
(i) a first fluid flow path defined between the pressurized gas source and the distal end of the insertion section, for delivery of insufflation gas from the pressurized gas source to a region of tissue of interest during an endoscopy procedure; and
(ii) a second fluid flow path defined between the pressurized gas source and the irrigation liquid reservoir, such that the irrigation liquid reservoir is pressurised by the pressurized gas source.

17. The imaging endoscope system according claim 16 wherein at least a part of the first and second fluid flow paths is the same.

18. The imaging endoscope system according claim 17 wherein the umbilical section connector comprises a flow path splitter arranged to split the first and second fluid flow paths.

19. The imaging endoscope system according to claim 16 wherein a third fluid flow path is-provided, defined between the irrigation liquid reservoir and the distal end of the insertion section, for delivery of irrigation liquid from the irrigation liquid reservoir to a region of tissue of interest during an endoscopy procedure.

20. A method for assembling an imaging endoscope system from a kit comprising:
a base unit;
a hand controller;
an umbilical section releasably connecting the base unit and the hand controller;
an insertion section having a proximal end connected to the hand controller and a distal end for insertion into a subject;
the base unit being configured for provision of electrical power and one or more fluids to the insertion section via the umbilical section and the hand controller; and
the umbilical section comprising an umbilical section connector configured to be releasably engageable with a corresponding base unit connector;
wherein the method comprises connecting the umbilical section connector and the base unit connector in a single connection action to thereby make at least one mechanical connection, at least one electrical connection, and at least one fluid connection between the umbilical section connector and the base unit connector in said single connection action.

21. The method according to claim 20 wherein the single connection action includes manually locating the umbilical section connector and the base unit connector into an engaged position.
